(19) United States
(12) Patent Application Publication
(10) Pub. No.: US 2017/0065297 A1
(43) Pub. Date: Mar. 9, 2017

(54) TRANSEPTAL NEEDLE WITH LOW TIP PRESSURE DESIGN

(71) Applicant: Medtronic Ablation Frontiers LLC, Minneapolis, MN (US)

(72) Inventor: Scott A. HARELAND, Lino Lakes, MN (US)

(21) Appl. No.: 14/844,305

(22) Filed: Sep. 3, 2015

(51) Int. Cl. A61B 17/34; A61B 17/00

(52) U.S. Cl. CPC A61B 17/3494; A61B 17/00234; A61B 2017/00247; A61B 2017/003; A61B 2017/00862

(57) ABSTRACT

A method and device for puncturing the atrial septum to gain access to the left atrium without causing unintended injury to left atrial tissue. Specifically, the device includes a transseptal needle having a compressible shaft and a puncturing tip. As the transseptal needle is advanced through a delivery sheath and into contact with the septum, the compressible shaft is compressed and stores a minimal amount of mechanical energy but allows force transfer along its length. Force continues to be applied to the needle by the user until the puncturing tip punctures the septum. As the puncturing tip advances through the puncture and into the left atrium, any mechanical energy stored in the compressible shaft is immediately released, and the transfer of force is discontinued, by physical deformation of the compressible shaft. Thus, the puncturing tip enters the left atrium without causing injury to left atrial wall tissue.

### CROSS-REFERENCE TO RELATED APPLICATION

n/a

### STATEMENT REGARDING FEDERALLY SPONSORED RESEARCH OR DEVELOPMENT

n/a

### TECHNICAL FIELD

The present invention relates to a method, system, and device for puncturing the atrial septum to gain access to the left atrium without causing unintended injury to left atrial tissue. Specifically, the device may include a transseptal needle having a compressible shaft and a puncturing tip. As the transseptal needle is advanced through a delivery sheath and into contact with the septum, the compressible shaft may be compressed and may store a minimal amount of mechanical energy but allows force transfer along its length. Force may continue to be applied to the needle by the user until the puncturing tip punctures the septum. As the puncturing tip advances through the puncture and into the left atrium, any mechanical energy stored in the compressible shaft may be immediately released, and the transfer of force may be discontinued, by physical deformation of the compressible shaft. Thus, the puncturing tip may enter the left atrium without causing injury to left atrial wall tissue

### BACKGROUND

Many cardiac treatment procedures require access to left atrium of the heart. For intravenous access, transseptal puncture is a critical step in gaining access to the left side of the heart. During transseptal puncture, a needle is passed through a delivery sheath 10 from the venous access point to the atrial septum, typically at the fossa ovalis. Force is applied to the needle near the external access point in order to build up pressure at the needle tip in contact with the cardiac tissue. Once the tip pressure is high enough to penetrate the septal wall, the needle passes through the septum and enters the left side of the heart, typically the left atrium.

The amount of force exerted from the needle tip on the septal wall can be significant. Consequently, a user may have difficulty in controlling the tip momentum as the septal wall is punctured and force continues to be transferred to the needle tip and the dilator. This applied force cannot be instantaneously removed as the tip penetration may result in a sudden “pop” through the tissue. The response time of the user is typically too slow to reduce the applied pressure at the moment the needle tip punctures the septal wall.

A risk of transseptal puncture is that the needle tip will unintentionally injure tissue at undesired locations in the heart. For example, if the applied force is not quickly reduced or removed at the time of puncture, the needle tip may continue in the direction of the applied force and come into contact with a wall of the left atrium.

Therefore, it is desired to provide a device, system, and method that reduce the risk of post-puncture cardiac damage when used to perform a transseptal procedure.

### SUMMARY

The present invention advantageously provides a device, system, and method for reducing the risk of post-puncture cardiac damage when used to perform a transseptal procedure. In particular, the present invention provides a transseptal device with a compressible shaft that permits force transfer in a sheath to penetrate tissue while rapidly dissipating tip pressure after puncture is achieved.

A transseptal device may include a needle having a compressible shaft with a proximal portion and a distal portion, the distal portion having a puncturing tip. The compressible shaft may be configured to compress when a force is applied to the proximal portion of the compressible shaft. Further, the needle may be configured to be advanced through a delivery sheath, the compressible coil being configured to be compressed when the needle is within a delivery sheath and uncompressed when the needle is advanced distally out of a delivery sheath. The puncturing tip may have a distalmost surface. For example, the distalmost surface of the puncturing tip may be tapered to a point configured to puncture septal tissue. Further, the puncturing tip may be composed of a low-mass material. The compressible shaft may be a spring or it may be composed of a deformable material having a low spring constant. The compressible shaft may be configured to transfer mechanical energy from the proximal portion to the puncturing tip, such as when the compressible shaft is compressed against an area of tissue, and may be configured to no longer transfer mechanical energy from the proximal portion to the puncturing tip immediately after the puncturing tip punctures the area of tissue.

A method of creating a transseptal puncture may include: advancing a compressible transseptal puncture needle into contact with an atrial septum such that the compressible transseptal puncture needle is compressed, the compressible transseptal puncture needle including a proximal portion and a distal portion and being configured to transfer mechanical energy from the proximal portion to the distal portion when compressed; puncturing the atrial septum with the compressible transseptal puncture needle; and discontinuing the transfer of mechanical energy by physical deformation of the compressible transseptal puncture needle. The compressible transseptal puncture needle may include a compressible shaft and a puncturing tip coupled to the compressible shaft. The compressible shaft may have a coiled configuration. The compressible shaft may be a helical compression spring. The compressible transseptal puncture needle may define a proximal portion and a distal portion and further includes a non-compressible segment at the proximal portion, and the puncturing tip may be coupled to the distal portion.

A method of creating a transseptal puncture may include: advancing a compressible transseptal puncture needle out a distal end of a delivery sheath and into contact with an atrial septum of a heart such that the compressible transseptal puncture needle is compressed, the compressible transseptal puncture needle being configured to transfer mechanical energy from a proximal portion to a distal portion of the transseptal puncture needle when compressed; puncturing the atrial septum with the compressible transseptal puncture needle; and immediately after puncturing the atrial septum with the compressed transseptal needle, discontinuing the transfer of mechanical energy by physically deforming the transseptal needle. The compressible transseptal puncture needle may include a compressible shaft and a puncturing tip coupled to the compressible shaft. The compressible shaft may have a coiled configuration. Further, the compressible transseptal puncture needle may define a proximal portion and a distal portion and further include a non-compressible segment at the proximal portion.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows an exemplary placement of a medical device within a heart with transseptal access to the left atrium;

FIGS. 2A and 2B show a prior art transseptal device being used to puncture the atrial septum;

FIG. 3 shows an exemplary graphical relationship between tip pressure and applied force when using a prior art transseptal device;

FIGS. 4A-4D show a transseptal device with a compressible needle shaft being used to puncture the atrial septum; and

FIG. 5 shows an exemplary graphical relationship between tip pressure and applied force when using the transseptal device with compressible needle shaft.

### DETAILED DESCRIPTION

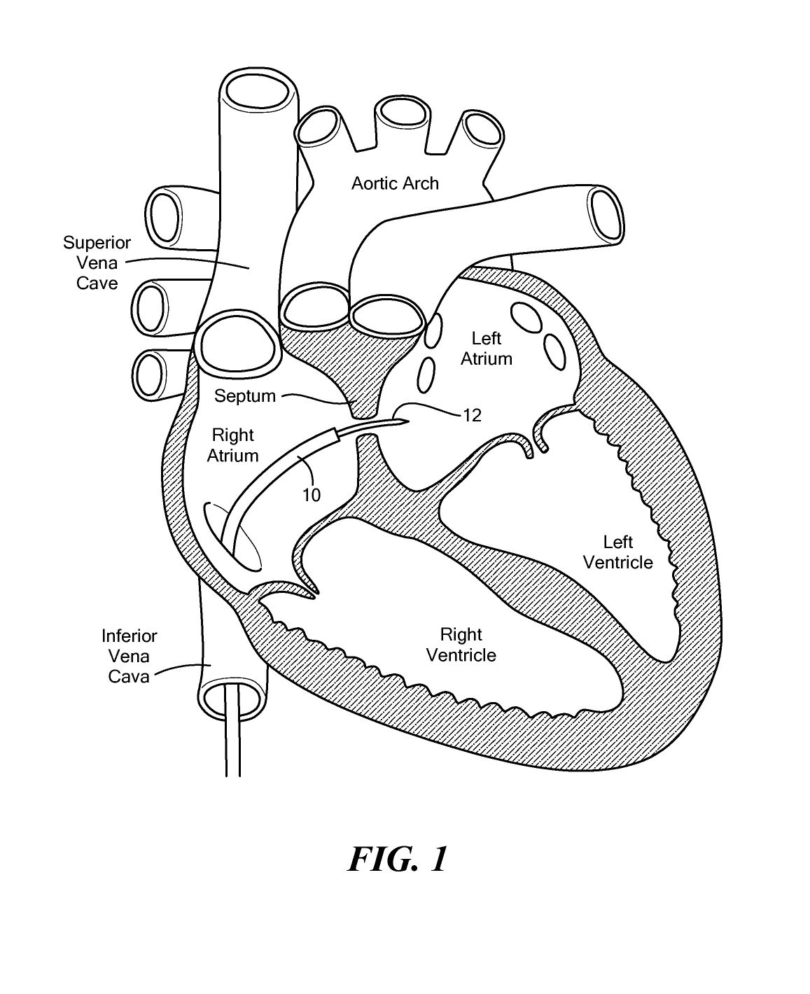

Referring now to FIG. 1, an exemplary placement of a medical device within a heart is shown. A delivery sheath 10 may be navigated to a patient's heart through the patient's vasculature, such as by femoral, radial, or brachial access. As a non-limiting example, the delivery sheath 10 may be navigated from the femoral artery, through the inferior vena cava, and into the right atrium, and the transseptal device 12 may be advanced through the delivery sheath 10 and into the heart. From the right atrium, the transseptal device 12 may be used to puncture the atrial septum, such as through the area of septal tissue known as the fossa ovalis, to gain access into the left atrium. The transseptal device 12 may include one or more puncture elements, such as a needle or cannula. Once the transseptal device 12 has punctured the atrial septum, the transseptal device 12 may be withdrawn through the delivery sheath 10 and removed from the heart, and a treatment device may be advanced through the delivery sheath 10 into the left atrium. Alternatively, the transseptal device 12 may also function as a treatment device, the transseptal device 12 remaining within the heart to treat cardiac tissue. Although other methods and treatment locations may be used, methods that require accessing left atrial tissue may involve puncturing the atrial septum with a transseptal device 12.

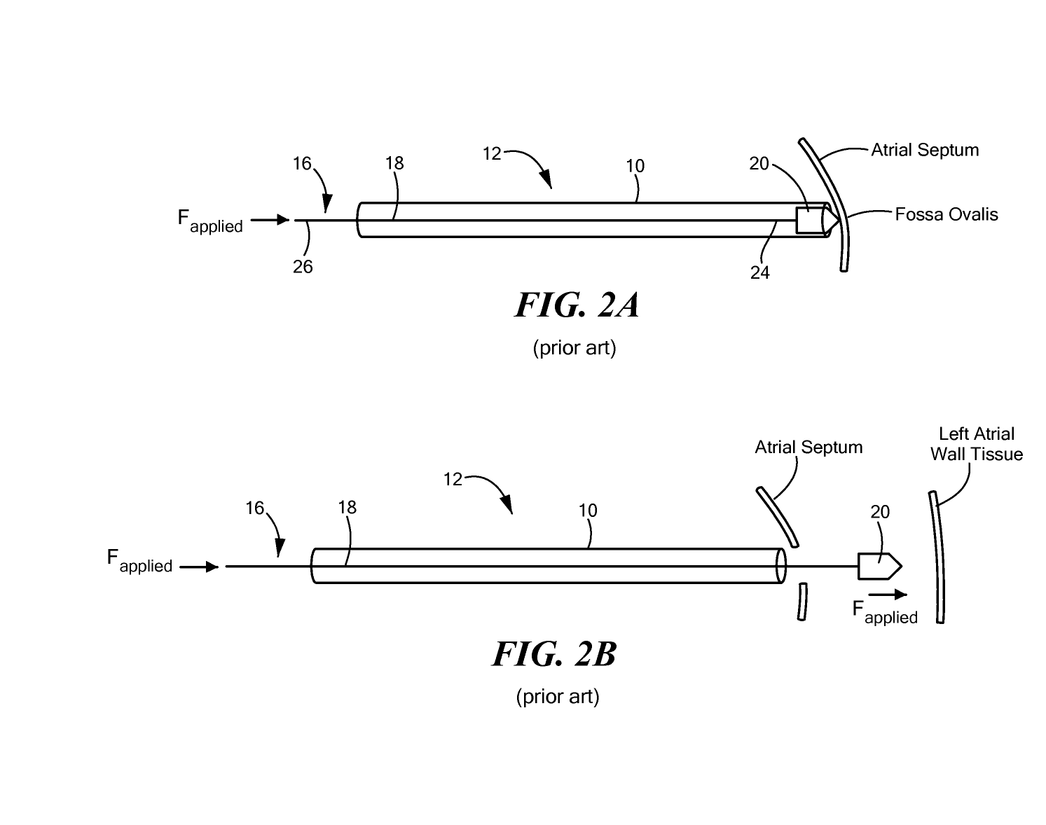

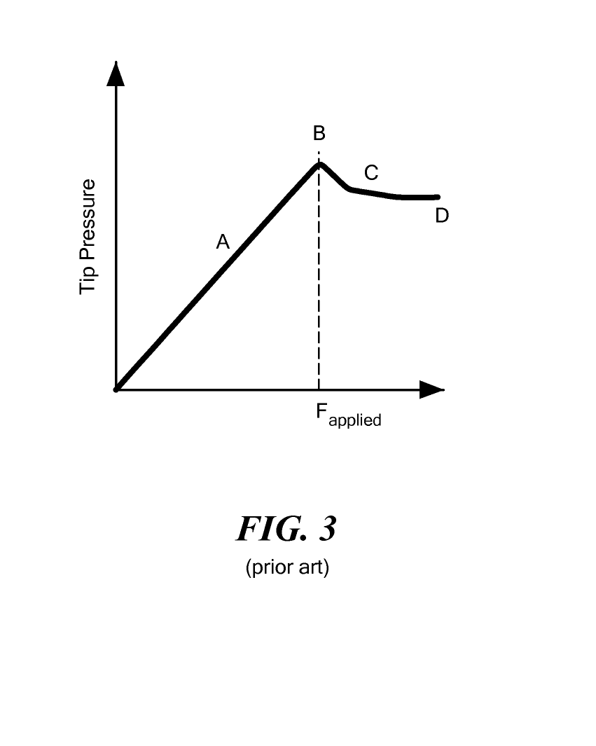

Referring now to FIGS. 2A-3, a prior art method of creating a transseptal puncture is shown. As is shown in FIGS. 2A and 2B, the transseptal device 12 may be advanced through a delivery sheath 10 to the atrial septum. For example, the transseptal device 12 may include a needle 16 with a shaft 18 and a puncturing tip 20. The shaft 18 may include a distal portion 24 and a proximal portion 26, with the puncturing tip 20 being at the distal portion 24 and the proximal portion 26 extending out of the patient and being mechanically operable by a user. For example, the user may apply force to the shaft 18 to advance the needle 16 in a forward direction. A force F_{applied} may be exerted along the shaft 18 to contact the atrial septum with the puncturing tip 20, and the force F_{applied} may be sufficient for the puncturing tip 20 to break through or puncture the septal tissue. However, the force F_{applied} required to puncture the septal tissue may be substantial, and therefore that force may continue to be exerted on the shaft 18 even after the septal tissue is punctured. This continued force may result in the puncturing tip 20 coming into contact with, and possibly injuring, the left atrial wall tissue downstream from the puncture site.

Even though the user may attempt to reduce or eliminate the force F_{applied} exerted on the shaft 18 immediately after the septal tissue is punctured, the user's reaction time may not be fast enough to prevent the puncturing tip 20 from contacting the left atrial wall tissue. As is shown graphically in FIG. 3, puncturing the atrial septum with currently known transseptal devices may include four stages. In the first stage A, the pressure of the puncturing tip 20 against the septum may increase as the amount of force F_{applied} applied to the shaft 18 is increased. In the second stage B, the puncturing tip 20 may penetrate the septal tissue. In the third stage C, the puncturing tip 20 may continue in a forward direction due to the continued applied force F_{applied} on the shaft 18. In the fourth stage D, the applied force F_{applied} may continue, even if somewhat reduced, and there is a risk that the puncturing tip 20 may injure left atrial wall tissue (or any other tissue within the heart other than the targeted septal tissue).

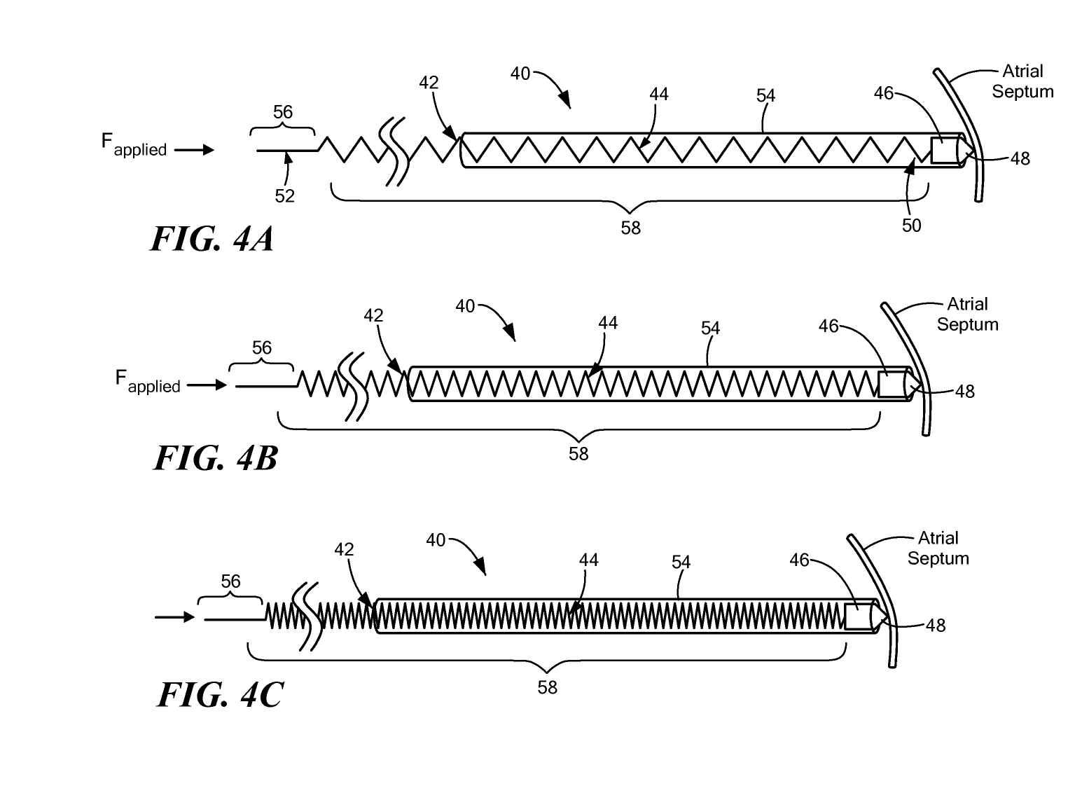

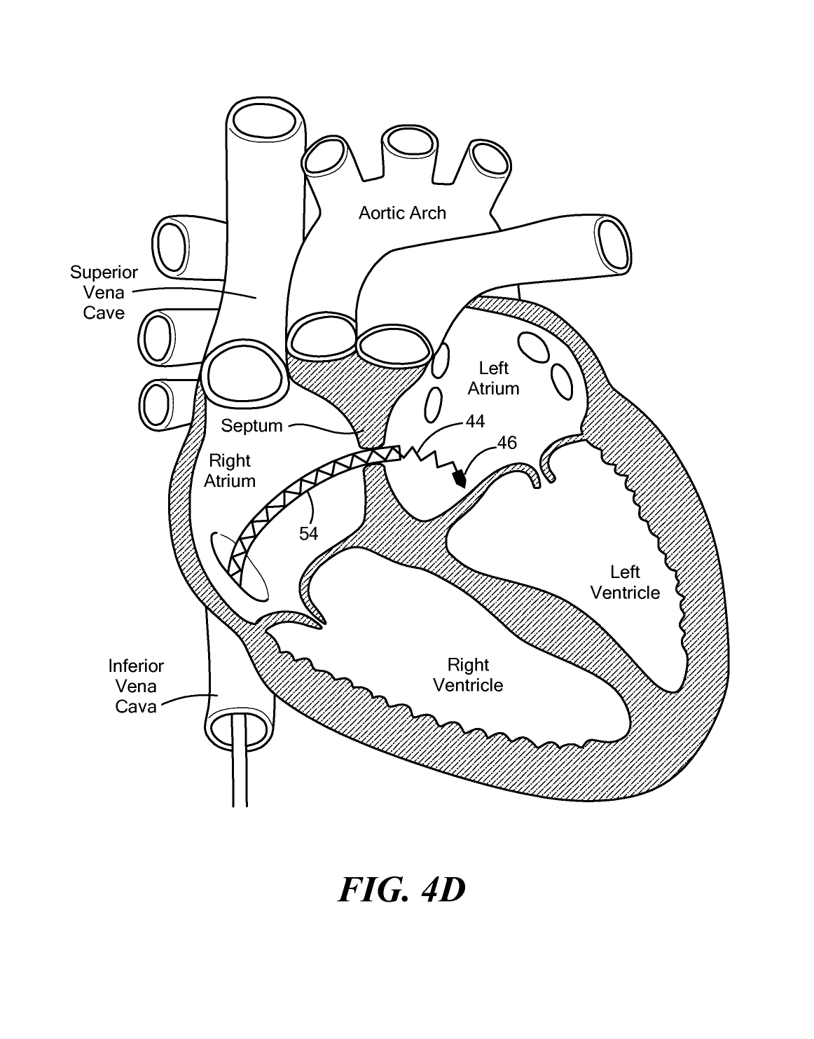

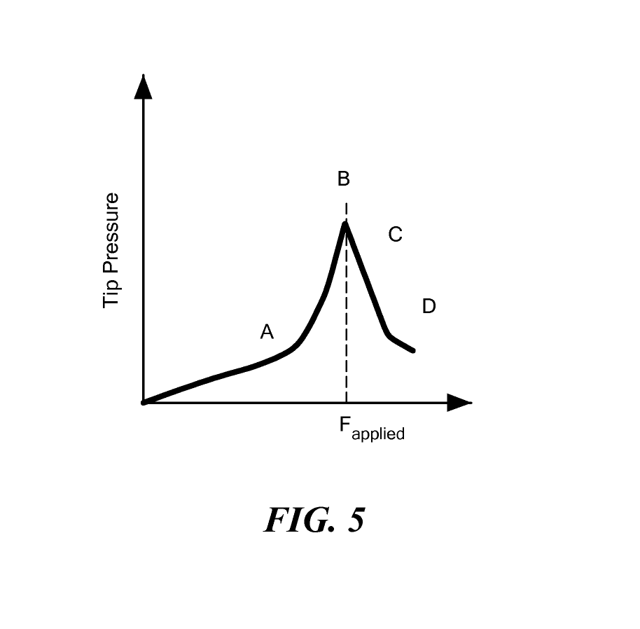

Referring now to FIGS. 4A-5, a method of creating a transseptal puncture with a transseptal device having a compressible needle shaft is shown. The transseptal device 40 may be navigated to a location proximate the atrial septum as shown and described in FIGS. 1-2B. Unlike the currently known transseptal device 12 of FIGS. 1-3, however, the transseptal device 40 of FIGS. 4A-5 may include a needle 42 with a compressible shaft 44 and a puncturing tip 46. The puncturing tip 46 may have a distalmost surface 48 that is sharp, pointed, tapered, or otherwise able to puncture tissue. The compressible shaft 44 may include a distal portion 50 and a proximal portion 52, with the puncturing tip 46 being at the distal portion 50 and the proximal portion 52 extending out of the patient and being mechanically operable by a user. As a non-limiting example, the puncturing tip 46 may be affixed to the distal portion 50 of the compressible shaft 44 by any known means, such as welding, laser welding, chemical or heat bonding, use of adhesives, or the like. Alternatively, the puncturing tip 46 may be integral with (for example, co-extruded with) the compressible shaft 44. The compressible shaft 44 may be a flexible coil, similar to a spring. In fact, the compressible shaft 44 may be a spring, such as a helical compression spring, that has a low spring constant in order to store a minimal amount of mechanical energy created by the force F_{applied} applied on the shaft 44. Although the compressible shaft 44 may be configured to store a minimal amount of mechanical energy, it may also be configured to facilitate transfer of the applied force F_{applied} from the rigid proximal portion 52 of the shaft 44 to the puncturing tip 46 when it is constrained within the delivery sheath 54. The coil design may be able to undergo compression while constrained within the delivery sheath 54 without directly applying a high level of pressure from the puncturing tip 46 to the septal tissue. Alternatively the compressible shaft 44 may not have a coiled configuration, but instead may be composed of a material that is physically deformable with a low spring constant such that only a small amount of energy is stored when it is physically compressed along the direction of the applied force.

As the user applies force F_{applied} on the proximal portion 52, the compressible shaft 44 may become compressed and pressure slowly builds up at the puncturing tip 46. Once the compressible shaft 44 becomes fully compressed within the delivery sheath 54, tip pressure may build between the puncturing tip 46 and the septal tissue as long as the compressible shaft 44 is constrained within the delivery sheath 54. Further, the proximal portion 52 of the compressible shaft 44 may include a rigid segment 56 at the proximalmost end of the shaft 44 that is not compressible and is able to transmit force F_{applied} along the shaft to the compressible segment 58.

When sufficient pressure at the puncturing tip 46 is reached, the puncturing tip 46 may break through or puncture the atrial septum. As the puncturing tip 46 and the compressible shaft 44 continue forward (i.e. in a proximal-to-distal direction) out of the distal end 60 of the delivery sheath 54 and through the puncture in the atrial septum, the compression of the compressible shaft 44 is immediately dissipated and the puncturing tip 46 “flops” forward into the left atrium without reaching the left atrial wall tissue (or other non-target tissue). That is, the compressible shaft 44 may be configured to release the stored mechanical energy and absorb the pressure from the proximal portion 52 via physical deformation immediately after the puncturing tip 46 punctures the area of tissue. As the compressible shaft 44 is no longer constrained within the delivery sheath 54, the transfer of applied force F_{applied} to the puncturing tip 46 is greatly diminished as shown in FIG. 4D. For example, a helical compression spring compressible shaft 44 may be fully compressed against the atrial septum and may expand to its non-compressed configuration immediately upon puncturing the septal tissue and entering the left atrium. To enhance the rapid dissipation of pressure, the puncturing tip 46 may be composed of a material having a low mass. Further, the compressible shaft 44 may be constructed from a variety of materials, including but not limited to stainless steel, and/or nitinol.

As is shown graphically in FIG. 5, puncturing the atrial septum with the transseptal device 40 with the compressible shaft 44 may include four stages. In the first stage A, the pressure at the puncturing tip 46 against the septum may increase gradually as the amount of force F_{applied} applied to the compressible shaft 44 is increased. The slope of the line shown in FIG. 5 may be less steep than the slope of the line in the first stage A shown in FIG. 3. That is, the pressure at the puncturing tip 46 of the transseptal device 40 shown in FIGS. 4A-4D may increase less rapidly than the pressure at the puncturing tip 20 of the transseptal device 12 shown in FIGS. 2A and 2B. In the second stage B, the puncturing tip 46 may penetrate the septal tissue. In the third stage C, the compression of the compressible shaft 44 may be immediately reduced, and force transfer discontinued, as the compressible shaft 44 relaxes and physically deforms (for example, the spring decompresses), and the puncturing tip 46 continues harmlessly into the left atrium. In the fourth stage D, the low-mass puncturing tip 46 does not transfer force F_{applied} to cardiac tissue within the left atrium.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A transseptal device, comprising:
a needle including a compressible shaft with a proximal portion and a distal portion, the distal portion having a puncturing tip.

2. The transseptal device of claim 1, wherein the compressible shaft is configured to compress when a force is applied to the proximal portion of the compressible shaft.

3. The transseptal device of claim 2, wherein the needle is configured to be advanced through a delivery sheath, the compressible shaft being configured to be compressed when the needle is within the delivery sheath and uncompressed when the needle is advanced distally out of the delivery sheath.

4. The transseptal device of claim 1, wherein the puncturing tip has a distalmost surface.

5. The transseptal device of claim 4, wherein the distalmost surface of the puncturing tip is tapered to a point configured to puncture septal tissue.

6. The transseptal device of claim 1, wherein the puncturing tip is composed of a low-mass material.

7. The transseptal device of claim 2, wherein the compressible shaft is a spring.

8. The transseptal device of claim 2, wherein the compressible shaft is composed of a deformable material having a low spring constant.

9. The transseptal device of claim 1, wherein the compressible shaft is configured to transfer mechanical energy from the proximal portion to the puncturing tip.

10. The transseptal device of claim 9, wherein the compressible shaft is configured to transfer mechanical energy when the compressible shaft is compressed against an area of tissue and is configured to no longer transfer mechanical energy from the proximal portion to the puncturing tip immediately after the puncturing tip punctures the area of tissue.

11. A method of creating a transseptal puncture, the method comprising:
advancing a compressible transseptal puncture needle into contact with an atrial septum such that the compressible transseptal puncture needle is compressed, the compressible transseptal puncture needle including a proximal portion and a distal portion and being configured to transfer mechanical energy from the proximal portion to the distal portion when compressed;
puncturing the atrial septum with the compressible transseptal puncture needle; and
discontinuing the transfer of mechanical energy by physical deformation of the compressible transseptal puncture needle.

12. The method of claim 11, wherein the compressible transseptal puncture needle includes:
a compressible shaft; and
a puncturing tip coupled to the compressible shaft.

13. The method of claim 12, wherein the compressible shaft has a coiled configuration.

14. The method of claim 12, wherein the compressible shaft is a helical compression spring.

15. The method of claim 12, wherein the compressible transseptal puncture needle defines a proximal portion and a distal portion and further includes a non-compressible segment at the proximal portion.

16. The method of claim 15, wherein the puncturing tip is coupled to the distal portion.

17. A method of creating a transseptal puncture, the method comprising:
advancing a compressible transseptal puncture needle out a distal end of a delivery sheath and into contact with an atrial septum of a heart such that the compressible transseptal puncture needle is compressed, the compressible transseptal puncture needle being configured to transfer mechanical energy from a proximal portion to a distal portion of the transseptal puncture needle when compressed;
puncturing the atrial septum with the compressible transseptal puncture needle; and
immediately after puncturing the atrial septum with the compressible transseptal needle, discontinuing the transfer of mechanical energy by physically deforming the transseptal needle.

18. The method of claim 17, wherein the compressible transseptal puncture needle includes:
a compressible shaft; and
a puncturing tip coupled to the compressible shaft.

19. The method of claim 18, wherein the compressible shaft has a coiled configuration.

20. The method of claim 19, wherein the proximal portion includes a non-compressible segment.
